(19) United States
(12) Patent Application Publication
(10) Pub. No.: US 2025/0009769 A1
(43) Pub. Date: Jan. 9, 2025

(54) USE OF LYSOPHOSPHATIDIC ACID (LPA) IN CONTROLLING PORCINE INFECTIOUS DIARRHEA

(71) Applicant: SICHUAN AGRICULTURAL UNIVERSITY, Yaan (CN)

(72) Inventors: Jie YU, Yaan (CN); Wenting DU, Yaan (CN); Daiwen CHEN, Yaan (CN); Hui YAN, Yaan (CN); Bing YU, Yaan (CN); Jun HE, Yaan (CN); Ping ZHENG, Yaan (CN); Xiangbing MAO, Yaan (CN); Aimin WU, Yaan (CN); Yueqi XUAN, Yaan (CN)

(21) Appl. No.: 18/598,622

(22) Filed: Mar. 7, 2024

Related U.S. Application Data
(63) Continuation of application No. PCT/CN2023/123562, filed on Oct. 9, 2023.

(30) Foreign Application Priority Data
Jul. 7, 2023 (CN) 202310830639.1

(51) Int. Cl. A61K 31/661; A23K 20/26; A23K 50/30; A61P 31/04

(52) U.S. Cl. CPC A61K 31/661; A23K 20/26; A23K 50/30; A61P 31/04

(57) ABSTRACT

The present disclosure provides use of lysophosphatidic acid (LPA) as a feed additive in controlling porcine infectious diarrhea, and belongs to the technical field of feed nutrition. The LPA is a type of lipid substance that is produced endogenously in vivo and can reduce a large secretion level of intestinal fluid in piglets caused by Escherichia coli infection via inhibiting cystic fibrosis transmembrane-conductance regulator (CFTR)-dependent iodine efflux. In this way, a susceptibility of weaned piglets to the Escherichia coli is prevented to effectively maintain the intestinal health of piglets. LPA is an endogenous substance in vivo, and it is first discovered that adding the LPA into a feed can control Escherichia coli-caused infectious diarrhea in the piglets.

### CROSS REFERENCE TO RELATED APPLICATION

This application is a national stage application of International Patent Application No. PCT/CN2023/123562, filed on Oct. 9, 2023, which claims priority to the Chinese Patent Application No. 202310830639.1, filed with the China National Intellectual Property Administration (CNIPA) on Jul. 7, 2023, which is incorporated herein by reference in its entirety.

### TECHNICAL FIELD

The present disclosure belongs to the technical field of feed nutrition, and relates to use of lysophosphatidic acid (LPA) as a feed additive in controlling porcine infectious diarrhea.

### BACKGROUND

Early weaning of piglets is one of the key technologies in modern intensive breeding, and can effectively shorten a lactation period, increase an annual productivity, and maximize a reproductive performance of sows. However, on one hand, piglets weaned early may lose the passive immunity from the mother's breast milk, which can reduce the piglet's immunity, making the piglet susceptible to exogenous pathogens and resulting in reduced disease resistance. On the other hand, the gastrointestinal tract of piglets is not fully developed during early weaning. At the same time, changes in intestinal appearance and structure may occur after weaning, including shortening of intestinal villi, increased crypt depth, and reduced digestive enzyme activity. These changes in the gastrointestinal tract can affect the digestion and absorption of nutrients in the diet by piglets. Moreover, undigested nutrients are easily exploited and reproduced by pathogenic bacteria after entering a back end of the intestinal tract to allow fermentation, thereby reducing a number of beneficial intestinal bacteria and increasing a number of harmful bacteria, such that the intestinal biological barrier is damaged. Meanwhile, when the intestinal permeability of piglets increases, exogenous pathogens in the intestinal tract can easily invade the body through intestinal epithelial cells, inducing intestinal inflammation, diarrhea and other symptoms in piglets.

According to the ability to produce enterotoxin during infection, the Escherichia coli can be divided into two categories: enterotoxigenic Escherichia coli (ETEC) and non-toxigenic Escherichia coli. ETEC is the most common pathogenic microorganism that causes intestinal diseases in livestock and poultry, and is also one of the important factors causing the death of piglets. ETEC rely on adhesion factors to colonize host intestinal epithelial cells. After massive proliferation, the ETEC destroys the intestinal microecological balance to cause secondary infection, and secretes enterotoxin that leads to an imbalanced osmotic pressure of intestinal epithelial cells. As a result, the intestinal barrier function is damaged to cause intestinal dysfunction, resulting in diarrhea, dehydration, and even death of piglets. When infecting piglet intestinal tracts, the ETEC binds to the host intestinal epithelium through adhesins. At present, the most studied animal-derived ETEC adhesins mainly include K88 (F4), K99 (F5), and 987P (F6), among which the K88 (F4) has attracted the most attention. The K88 mainly includes three serotypes: K88ab, K88ac, and K88ad. The K88ac is considered to be the main serotype causing diarrhea in piglets, and infection of piglet intestinal epithelial cells (IPEC-J2) by ETEC K88ac can lead to the most pronounced immune response. After colonization in the small intestine, ETEC can proliferate in large quantities and secrete the virulence factor enterotoxin directly related to diarrhea, and the enterotoxin includes two of heat-labile enterotoxin (LT) and heat-stable enterotoxin (ST). ST secreted by porcine ETEC can bind to the extracellular domain of guanylate cyclase C (GC-C) and sulfide receptors in porcine intestinal epithelial cells, leading to an imbalance of water and salt metabolism in the intestine, such that a large amount of water and electrolytes extravasate, causing secretory diarrhea.

During the production, when piglets develop infectious diarrhea caused by Escherichia coli, the infected piglets are generally treated with antibiotics. However, while treating diarrheal diseases, the antibiotics can cause toxic side effects such as reduced immunity of livestock and poultry, increased bacterial resistance, drug residues, genetic mutations, deformities, and cancer.

Lysophosphatidic acid (LPA) is a biologically-active glycerophospholipid found in high concentrations in serum but in low concentrations in other eukaryotic tissues. The LPA is known to have at least six high-affinity, homologous rhodopsin-like G-protein coupled receptors, as well as one nuclear receptor and peroxisome proliferator-activated receptor C (PPARc). The LPA has three broad categories of biological effects. The first is normal cell functions, such as smooth muscle contraction, cell autotaxis, Ca²⁺ mobilization and neurotransmitter release, platelet aggregation, erythropoiesis, hematopoiesis of the bone marrow matrix, normal cell proliferation and migration, and cell differentiation. The second is to inhibit various diseases, including schizophrenia, atherosclerosis, abnormal itching, asthma, fibrosis, reproductive disorders, and bone metabolism disorders. The third is to regulate the acquisition of malignant abilities for multiple cancer cell lines, such as abnormal proliferation and migration, invasion, autophagy, and drug resistance of cancer cells.

### SUMMARY

An objective of the present disclosure is to provide use of LPA as a feed additive in controlling porcine infectious diarrhea, where the LPA (1-oleoyl-sn-glycerol 3-phosphate sodium salt) has a molecular weight of 510.39 and a structure as follows:

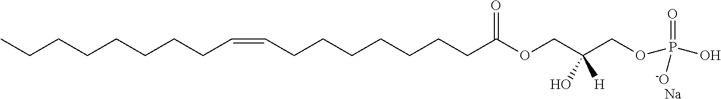

Preferably, the porcine infectious diarrhea is caused by Escherichia coli.

Preferably, the feed additive is in a form of an oral powder or a granule.

Preferably, the feed additive is a composition and a mixture with an effective dose of the LPA.

Preferably, the composition of the feed additive is a premix prepared by mixing the LPA with a carrier.

Preferably, the premix of the LPA is added at 0.1% to 0.5% of a total weight of a feed.

Compared with the prior art, the present disclosure has the following beneficial effects:

The LPA is a type of lipid substance that is produced endogenously in vivo and can reduce a large secretion level of intestinal fluid in piglets caused by Escherichia coli infection via inhibiting cystic fibrosis transmembrane-conductance regulator (CFTR)-dependent iodine efflux. Meanwhile, a susceptibility of weaned piglets to the Escherichia coli is prevented to effectively maintain the intestinal health of piglets. LPA is an endogenous substance in vivo, and it is first discovered that adding the LPA into a feed can control Escherichia coli-caused infectious diarrhea in the piglets. Moreover, the LPA has no toxic or side effects, such that animal excrement does not pollute the environment.

### DETAILED DESCRIPTION OF THE EMBODIMENTS

### Example 1

### 1. Experimental Materials

25-day-old DLY weaned piglets were selected, with an average weight of (7.24±0.07) kg.

### 2. Experimental Design

In the experiment, 32 piglets were divided into 4 groups with 8 replicates in each group, and the experimental period was 21 d. The diets of an ETEC+LPA group and an LPA group were supplemented with 0.3% LPA; on an 18th day of the experiment, a control group and the LPA group were fed 500 mL of a physiological saline, and the piglets in an ETEC group and the ETEC+LPA group were fed 500 mL of an ETEC bacterial solution; where ETEC had a concentration of 1×10⁹ CFU. The specific grouping was shown in Table 1. Slaughtering and sampling were conducted on the morning of day 21 of the experiment.

TABLE 1
Experimental Grouping

| Treatment | | | | ETEC + |
|---|---|---|---|---|
| group | CON group | ETEC group | LPA group | LPA group |
| ETEC | − | + | − | + |
| LPA | − | − | + | + |

### 3. Experimental Results

### 3.1. Effect of LPA on the Rate of Infectious Diarrhea Rate Caused by ETEC in Piglets

As shown in Table 2, ETEC challenge significantly increased the diarrhea rate and diarrhea index of piglets (P<0.05). Dietary LPA could significantly improve piglet diarrhea caused by ETEC and significantly reduce the diarrhea rate (P<0.05) and diarrhea index of piglets (P<0.05).

TABLE 2
Effects of dietary LPA on diarrhea indicators in piglets after ETEC challenge

| | Treatment | | | | P value | | |
|---|---|---|---|---|---|---|---|
| Item | CON | LPA | ETEC | LPA + ETEC | LPA | ETEC | L × E |
| Diarrhea rate (%) | 5.50 ± 13.47^{c} | 0.00 ± 0.00^{c} | 87.50 ± 24.87^{a} | 37.50 ± 27.99^{b} | <0.01 | <0.01 | 0.02 |
| Diarrhea index | 0.11 ± 0.27^{c} | 0.00 ± 0.00^{c} | 1.79 ± 0.53^{a} | 0.75 ± 0.56^{b} | <0.01 | <0.01 | 0.01 |

Note: Within a row, mean values of different letter superscripts were significantly different (P<0.05). Same for the tables below.

3.2. Effect of LPA on Intestinal Tissue Morphology of Piglets Infected with ETEC

As shown in Table 3, there was no significant difference in villus height, crypt depth, and villus height/crypt depth ratio (V/C) between the CON group and the LPA group (P>0.05). ETEC challenge reduced villus height and V/C in the duodenum and ileum (P<0.05), and increased crypt depth in the ileum (P<0.05). Dietary LPA significantly increased the villus height and V/C in the duodenum and ileum of piglets challenged with ETEC (P<0.05). Furthermore, the LPA reduced the crypt depth and increased the villus height/crypt depth (V/C) ratio in the ileum of ETEC-challenged piglets (P<0.05).

TABLE 3
Effects of dietary LPA on intestinal tissue morphology of piglets

| | Treatment | | | | P value | | |
|---|---|---|---|---|---|---|---|
| Item | CON | LPA | ETEC | LPA + ETEC | LPA | ETEC | L × E |
| Duodenum | | | | | | | |
| Villus height (μm) | 466.34 ± 13.21^{a} | 493.34 ± 12.43^{a} | 369.78 ± 12.98^{c} | 452.55 ± 11.68^{b} | 0.03 | 0.03 | 0.15 |
| Crypt depth (μm) | 276.32 ± 9.98 | 270.03 ± 8.13 | 298.95 ± 10.12 | 273.45 ± 9.78 | 0.17 | 0.14 | 0.23 |
| V/C ratio | 1.69 ± 1.32^{b} | 1.83 ± 1.53^{a} | 1.24 ± 1.28^{c} | 1.65 ± 1.19^{b} | 0.02 | 0.03 | 0.24 |
| Jejunum | | | | | | | |
| Villus height (μm) | 389.12 ± 10.23 | 377.28 ± 9.89 | 397.98 ± 9.28 | 360.56 ± 10.87 | 0.52 | 0.47 | 0.42 |
| Crypt depth (μm) | 175.04 ± 4.34 | 160.98 ± 3.23 | 180.78 ± 4.25 | 177.98 ± 6.30 | 0.86 | 0.79 | 0.60 |
| V/C ratio | 2.22 ± 2.36 | 2.34 ± 3.06 | 2.20 ± 2.18 | 2.02 ± 1.73 | 0.78 | 0.89 | 0.76 |
| Ileum | | | | | | | |
| Villus height (μm) | 390.87 ± 15.64^{a} | 401.46 ± 13.45^{a} | 330.34 ± 12.89^{b} | 378.37 ± 13.76^{a} | 0.04 | 0.03 | 0.12 |
| Crypt depth (μm) | 169.76 ± 3.09^{b} | 150.45 ± 4.89^{b} | 210.56 ± 5.02^{a} | 170.08 ± 4.12^{b} | 0.02 | 0.04 | 0.25 |
| V/C ratio | 2.30 ± 5.06^{b} | 2.67 ± 2.75^{a} | 1.57 ± 2.57^{c} | 2.22 ± 3.34^{b} | 0.02 | 0.01 | 0.11 |

3.3. Effect of LPA on Intestinal Permeability of Piglets Infected with ETEC

As shown in Table 4, compared with the CON group, the LPS, DAO, D-lactic acid, and fluid accumulation rate of ETEC-challenged piglets were increased significantly (P<0.05); Dietary LPA significantly reduced the LPS, DAO, D-lactic acid, and fluid accumulation rate of the ETEC-challenged piglets (P<0.05).

TABLE 4
Effects of dietary LPA on permeability of piglets after ETEC challenge

| | Treatment | | | | P value | | |
|---|---|---|---|---|---|---|---|
| Item | CON | LPA | ETEC | LPA + ETEC | LPA | ETEC | L × E |
| LPS (ng/L) | 47.56 ± 6.78^{b} | 48.23 ± 5.23^{b} | 60.12 ± 4.33^{a} | 49.13 ± 2.56^{b} | 0.04 | 0.03 | 0.23 |
| DAO (pg/mL) | 321.45 ± 14.54^{b} | 330.43 ± 12.78^{b} | 436.89 ± 11.09^{a} | 335.62 ± 14.23^{b} | 0.03 | 0.02 | 0.13 |
| D-lactic acid (mg/L) | 1.65 ± 0.89^{b} | 1.69 ± 0.22^{b} | 2.01 ± 0.54^{a} | 1.53 ± 0.93^{b} | 0.05 | 0.04 | 0.21 |
| Fluid accumulation rate | 1.22 ± 0.34^{b} | 1.32 ± 1.21^{b} | 1.79 ± 0.78^{a} | 1.42 ± 0.68^{b} | 0.04 | 0.03 | 0.18 |

### 4. Conclusion

LPA had no significant negative impact on piglet growth performance, could significantly prevent the impairment on intestinal morphology and reduced permeability caused by ETEC, and could significantly reduce the diarrhea rate caused by Escherichia coli in piglets.

### Example 2

### 1. Experimental Materials

21-day-old DLY weaned piglets were selected, with an average weight of (6.23±0.82) kg.

### 2. Experimental Design

In the experiment, 24 piglets were divided into 3 groups, each group had 8 replicates, and the experimental period was 21 d. The diets of an ETEC+LPA group were supplemented with 0.2% LPA; on an 18th day of the experiment, a control group was fed 200 mL of saline, and the piglets in an ETEC group and the ETEC+LPA group were fed 200 mL of an ETEC bacterial solution; where ETEC had a concentration of 1×10⁹ CFU. The specific grouping was shown in Table 5. Slaughtering and sampling were conducted on the morning of day 21 of the experiment.

TABLE 5
Experimental Grouping

| Treatment group | Control | ETEC group | ETEC + LPA group |
|---|---|---|---|
| ETEC | − | + | + |
| LPA | − | − | + |

### 3. Experimental Results

As shown in Table 6, LPA added before challenge could significantly reduce the diarrhea rate (P<0.05). At the same time, the diarrhea rate and diarrhea index of Escherichia coli-challenged piglets added with LPA after challenge also decreased significantly (P<0.05).

TABLE 6
Diarrhea rate caused by Escherichia coli in piglets

| Item | Control | ETEC group | ETEC + LPA group | P value |
|---|---|---|---|---|
| Before challenge | | | | |
| Diarrhea rate (%) | 15.21 ± 0.21^{a} | 16.09 ± 0.13^{a} | 11.86 ± 0.15^{b} | 0.04 |
| Diarrhea index | 1.73 | 1.84 | 1.68 | 0.89 |
| After challenge | | | | |
| Diarrhea rate (%) | 13.23 ± 0.13^{c} | 89.32 ± 0.34^{a} | 33.56 ± 0.28^{b} | <0.01 |
| Diarrhea index | 1.23^{c} | 2.92^{a} | 1.98^{b} | 0.05 |

### 4. Conclusion

Dietary 0.2% LPA could effectively reduce the diarrhea rate caused by Escherichia coli in weaned piglets.

The above examples are only intended to describe the preferred implementations of the present disclosure, but not to limit the scope of the present disclosure. Various alterations and improvements made by those of ordinary skill in the art based on the technical solution of the present disclosure without departing from the design spirit of the present disclosure shall fall within the scope of the appended claims of the present disclosure.

What is claimed is:

1. A method for controlling porcine infectious diarrhea, comprising: controlling the porcine infectious diarrhea by using lysophosphatidic acid (LPA) as a feed additive.

2. The method according to claim 1, wherein the porcine infectious diarrhea is caused by Escherichia coli.

3. The method according to claim 1, wherein the feed additive is in a form of an oral powder or a granule.

4. The method according to claim 2, wherein the feed additive is in a form of an oral powder or a granule.

5. The method according to claim 1, wherein the feed additive is a composition and a mixture with an effective dose of the LPA.

6. The method according to claim 2, wherein the feed additive is a composition and a mixture with an effective dose of the LPA.

7. The method according to claim 5, wherein the composition of the feed additive is a premix prepared by mixing the LPA with a carrier.

8. The method according to claim 6, wherein the composition of the feed additive is a premix prepared by mixing the LPA with a carrier.

9. The method according to claim 7, wherein the premix of the LPA is added at 0.1% to 0.5% of a total weight of a feed.

10. The method according to claim 8, wherein the premix of the LPA is added at 0.1% to 0.5% of a total weight of a feed.
